(19) United States
(12) Patent Application Publication
(10) Pub. No.: US 2020/0149103 A1
(43) Pub. Date: May 14, 2020

(54) METHODS AND DEVICES FOR SAMPLE COLLECTION AND ANALYSIS

(71) Applicant: Cradle Genomics, Inc., San Diego, CA (US)

(72) Inventors: Sascha Drewlo, Grand Rapids, MI (US); Tristan Orpin, San Diego, CA (US); D. Randall Armant, San Diego, CA (US)

(21) Appl. No.: 16/743,970

(22) Filed: Jan. 15, 2020

Related U.S. Application Data
(63) Continuation-in-part of application No. PCT/US2019/012661, filed on Jan. 8, 2019.
(60) Provisional application No. 62/614,692, filed on Jan. 8, 2018.
(60) Provisional application No. 62/614,691, filed on Jan. 8, 2018.

(51) Int. Cl. C12Q 1/6869; G01N 1/28; A61B 10/02

(52) U.S. Cl. CPC C12Q 1/6869; A61B 10/0291; G01N 1/28

(57) ABSTRACT

The present invention is directed to methods of removing non-target DNA contamination from sample. The invention additionally is directed to the analysis of fetal DNA from an endocervical sample. The present invention is also directed to methods and devices for collection of an endocervical sample from a subject and subsequent analysis of the sample for diagnostic purposes.

### CROSS-REFERENCE TO RELATED APPLICATIONS

This application is a continuation-in-part of International Application No. PCT/US2019/012661, filed Jan. 8, 2019, now pending, which claims the benefit of priority under 35 U.S.C. § 119(e) of U.S. Patent Application Ser. Nos. 62/614,692 and 62/614,691, both filed Jan. 8, 2018. The disclosures of the prior applications are considered part of and are incorporated by reference in the disclosure of this application in their entireties.

### BACKGROUND OF THE INVENTION

### Field of the Invention

The present invention relates generally to methods and devices for collecting an endocervical sample from a subject, and more specifically to methods and devices for collecting an endocervical sample from a subject and subsequent analysis of the sample for diagnostic purposes.

### Background Information

DNA isolation is an established process in molecular biology. During the process, cells are lysed as a whole and DNA bound to a matrix, or differential solubility in organic and inorganic solvents is used to remove cell material such as proteins and other components unwanted in a clean DNA sample.

Under certain circumstances foreign and/or unwanted DNA (e.g., viruses/bacteria/cell free DNA) can cohabitate with a target cell. This DNA can enter or stick to the target cell population interfering with down-stream DNA based analyses such as PCR, sequencing, and whole genome amplification. This is particularly challenging if the target DNA to be analyzed exists in both the contaminating DNA host as well as in the cell type of interest. This situation requires the target DNA to have a certain amount of the total fraction to be analyzed precisely. In this case, the contaminating DNA would compete with the target DNA and mask the signal, making analysis challenging to impossible.

Previous attempts at nuclei isolation have proven unsuccessful in automated and high-throughput systems used in industry. Further, the needed reproducibility does not exist.

Currently, there is no method or kit available that allows for the efficient removal of contaminating DNA (e.g., extranuclear DNA, maternal DNA, microbial DNA, viral DNA or cell-free DNA) in a cell of interest using a nuclei isolation on DNA matrix approach that can be used for both manual and high- throughput applications.

### SUMMARY OF THE INVENTION

The present invention is based on the seminal discovery that fetal cells can be isolated from an endocervical sample from a pregnant subject. The invention further includes the isolation of target nucleic acid (e.g., fetal nucleic acid) from an endocervical sample containing target and non-target nucleic acid and the subsequent analysis of the target nucleic acid.

In one embodiment, the present invention provides a method of isolating target nucleic acid from a sample comprising cells by incubating the cells from the sample on a DNA binding membrane or a DNA binding matrix with a protein cocktail containing at least one enzyme to free the cellular nuclei; washing the DNA binding membrane or DNA binding matrix to remove non-target nucleic acid; lysing the nuclei to release the target nucleic acid; and isolating the target nucleic acid. In one aspect the target nucleic acid is fetal nucleic acid and the non-target nucleic acid is maternal nucleic acid, viral nucleic acid, microbial nucleic acid or cell free DNA. In an additional aspect, the cells are human. In certain aspects, the cells are maternal and/or fetal cells. In one aspect, the sample is an endocervical sample and the endocervical sample comprises maternal and fetal cells. In certain aspects, the sample comprises about 1-10 cells, 10-25 cells, 25-50 cells, 50-100 cells, 100-250 cells, 25-500 cells, 500-750 cells, 750-1000 cells, 1000-2500 cells or 2500-5000 cells. In one aspect, the cells are not fixed or bound to a surface (e.g., membrane or matrix, either DNA binding or non-DNA binding). In an additional aspect, the endocervical sample is collected using a menstruation cup. In a further aspect, the protein cocktail comprises a proteinase that preferably digests cellular walls but does not digest the nuclear envelope. In certain aspects, the protein cocktail comprises pepsin and preferably does not comprise DNase. If a sampling condition is chosen that does not allow free flow of ions in and out of the cells (e.g., live cells) although less controlled a hypotonic solution could be used to free the nuclei from other cellular material. In one aspect, the cells are incubated with the protein cocktail under non-binding conditions. In another aspect, lysing the nuclei comprises incubating the cells with a lysis buffer. In certain aspects, the lysis buffer is an enzymatic or a non-enzymatic lysis buffer. In certain aspects, the lysis buffer comprises proteinase K and/or trypsin. In an additional aspect, the target nucleic acid binds to the DNA binding membrane or DNA binding matrix. In a further aspect, isolating the target nucleic acid comprises eluting the nucleic acid from the DNA binding membrane or DNA binding. In one aspect, non-target nucleic acid contamination of the isolated target nucleic acid is less than about 1%, 2%, 3%, 4%, 5%, 10%, 15%, 20% or less than about 50%. In a further aspect, isolated target nucleic acid is analyzed by DNA sequencing, PCR or whole genome amplification.

In an additional embodiment, the present invention provides a method of analyzing fetal nucleic acid from an endocervical sample comprising isolating fetal cells from the endocervical sample; incubating the fetal cells on a DNA binding membrane or a DNA binding matrix with a protein cocktail containing at least one enzyme to free the cellular nuclei; washing the DNA binding membrane or DNA binding matrix remove non-target nucleic acid; lysing the nuclei to release the fetal nucleic acid; and isolating the fetal nucleic acid. In one aspect, the endocervical sample is collected using a menstrual cup. In another aspect, the endocervical sample comprises maternal and fetal cells. In an additional aspect, isolating the fetal cells comprises binding of the fetal cells to an anti-HLA antibody. In certain aspects, the sample comprises about 1-10 cells, 10-25 cells, 25-50 cells, 50-100 cells, 100-250 cells, 25-500 cells, 500-750 cells, 750-1000 cells, 1000-2500 cells or 2500-5000 cells. In one aspect, the cells are not fixed or bound to a surface (e.g., membrane or matrix, either DNA binding or non-DNA binding). In a further aspect, the protein cocktail comprises a proteinase that preferably digests cellular walls but does not digest the nuclear envelope. In certain aspects, the protein cocktail comprises pepsin, and preferably does not comprise DNase. In one aspect, lysing the nuclei comprises incubating the cells with a lysis buffer. In certain aspects, the lysis buffer is an enzymatic or a non-enzymatic lysis buffer. In certain aspects, the lysis buffer comprises proteinase K and/or trypsin. In another aspect, the released fetal nucleic acid binds to the DNA binding membrane or DNA binding matrix. In an additional aspect, isolating the fetal nucleic acid comprises eluting the nucleic acid from the DNA binding membrane or DNA binding matrix. In certain aspects, non-target nucleic acid contamination of the fetal nucleic acid is less than about 1%, 2%, 3%, 4%, 5%, 10%, 15%, 20% or less than about 50%. In a further aspect, the isolated fetal nucleic acid is analyzed by DNA sequencing, PCR or whole genome amplification. In one aspect, analyzing the fetal nucleic acid comprises identifying a genetic anomaly or gene based disease; a gene mutation; or chromosomal abnormality. In an additional aspect, analyzing the fetal nucleic acid comprises identifying a disease or condition resulting from a genetic anomaly, a gene mutation, or chromosomal abnormality is achondroplasia, Down syndrome, trisomy 21, trisomy 18, trisomy 13, Turner syndrome, Sickle cell disease, Cystic fibrosis, fragile XD syndrome, Muscular dystrophy, Tay-Sachs disease, spina bifida, anencephaly, Thalassemia, Polycystic kidney disease, Hemophilia A, Huntington's disease, or congenital adrenal hyperplasia.

In a further embodiment, the invention provides for a kit for the collection of an endocervical sample comprising a foldable menstruation cup; a storage container; and transport media. In one aspect, the menstruation cup is inserted into the vaginal canal. In another aspect, the menstruation cup is inserted for a time and under conditions to allow for sample collection, for example, for about 10 minutes, 15, minutes, 20 minutes, 25 minutes, 30 minutes, 35 minutes, 40 minutes, 45 minutes, 50 minutes, 55 minutes, 60 minutes, less than one hour, 1-2 hours, 1-5 hours, 1-10 hours, 1-20 or more hours. In an additional aspect, the transport media comprises at least one cell preservation chemical. In a further aspect, the preservation chemical is glycerol, serum, dimethyl sulfoxide, methanol, acetic acid, cell culture medium, a desiccation agent or a combination thereof. In another aspect, the sample is collected without a transport medium.

The present invention is also based on a collection device and method for collecting an endocervical sample a subject. The device is configured to collect the endocervical sample directly from a cervical canal by being placed at a location proximal to an external os of a uterine cervix or anywhere within the vaginal canal of the subject. The invention further includes analysis of the sample.

In yet another embodiment, the invention provides a method for conducting an assay. The method includes conducting an assay to detect an analyte present in an endocervical sample from a subject, wherein the sample is collected into a disposable resiliently deformable and flexible cup or disc disposed at a location proximal to an external os or anywhere within the vaginal canal of a uterine cervix of the subject. In one aspect, the method further includes isolating the analyte from the endocervical sample and optionally analyzing the analyte. In an aspect, analysis includes detecting a disease or condition associated with the subject, including for example, cancer, sexually transmitted infection, pregnancy, fetal defect, status of microbiome or presence of a microorganism. In an aspect, analysis includes detecting a marker for female reproductive management, such as miscarriage, fetal growth restriction, preterm labor, ruptured membrane, or any combination thereof. In an aspect, analysis includes detecting a marker for female reproductive oncology, such as cancer, including but not limited to ovarian, uterine, or cervical cancer. In an aspect, analysis includes detecting a marker of a female reproductive disorder, such as endometriosis or polycystic ovarian syndrome. In an aspect, analysis includes detection of an infection, such as a uterine infection. In an aspect, analysis includes detecting the presence and strain type of a virus, such as human papillomavirus. In certain aspects, the analyte is a cell or portion thereof, or cellular nuclei or portion thereof. In certain aspects, the analyte is a biomolecule, such as a nucleic acid, amino acid, protein, peptide, hormone, steroid, lipid, carbohydrate and ion. In certain aspects, the analyte is DNA and/or RNA, such as mRNA, tRNA or miRNA. In certain aspects, the analyte is a chemical compound. In certain aspects, the analyte is a microorganism, such as a virus, bacteria or fungi, or portion thereof. In one aspect, the analyte is human chorionic gonadotropin.

In still another embodiment, the invention provides a method of collecting an endocervical sample and analyzing the sample. The method includes: a) disposing a disposable resiliently deformable and flexible cup or disc at a location proximal to an external os or anywhere within the vaginal canal of a uterine cervix of a subject; b) collecting an endocervical sample into the disposable resiliently deformable and flexible cup or disc while the cup or disc is located proximal to the external os or anywhere within the vaginal canal; c) removing the cup or disc containing the endocervical sample from the subject; and d) conducting an assay to detect an analyte present in the endocervical sample. In one aspect, steps (a)-(c) are performed by the subject. In another aspect, steps (a)-(d) are performed by the subject. In another aspect, steps (a)-(c) are performed in an at-home setting and (d) is performed by a clinician other than the subject in a clinical setting. In one aspect, the method further includes isolating the analyte from the endocervical sample and optionally analyzing the analyte. In an aspect, analysis includes detecting a disease or condition associated with the subject, including for example, cancer, sexually transmitted infection, pregnancy, fetal defect, status of microbiome or presence of a microorganism. In an aspect, analysis includes detecting a marker for female reproductive management, such as miscarriage, fetal growth restriction, preterm labor, ruptured membrane, or any combination thereof. In an aspect, analysis includes detecting a marker for female reproductive oncology, such as cancer, including but not limited to ovarian, uterine, or cervical cancer. In an aspect, analysis includes detecting a marker of a female reproductive disorder, such as endometriosis or polycystic ovarian syndrome. In an aspect, analysis includes detection of an infection, such as a uterine infection. In an aspect, analysis includes detecting the presence and strain type of a virus, such as human papillomavirus. In certain aspects, the analyte is a cell or portion thereof, or cellular nuclei or portion thereof. In certain aspects, the analyte is a biomolecule selected from the group consisting of a nucleic acid, amino acid, protein, peptide, hormone, steroid, lipid, carbohydrate and ion. In certain aspects, the analyte is DNA and/or RNA, such as mRNA, tRNA or miRNA. In certain aspects, the analyte is a chemical compound. In certain aspects, the analyte is a microorganism, such as a virus, bacteria or fungi, or portion thereof. In one aspect, the analyte is human chorionic gonadotropin.

In yet another embodiment, of the invention provides a device for collecting an endocervical sample. The device includes a ring having a central opening; and a membrane coupled to the ring covering the central opening, the membrane having a reagent for performing an assay deposited thereon, wherein the device is resiliently deformable and flexible. In one aspect, the ring is approximately circular or oval in a non-deformed position. The ring may be sized to be disposed over or proximal to an external os of a uterine cervix or anywhere within the vaginal canal of a human subject. In certain aspects, the device is composed of a biocompatible polymer. In another aspect, the ring is composed of a first biocompatible polymer and the membrane is composed of a second biocompatible polymer. In another aspect, the ring and the membrane are composed of the same biocompatible polymer. In various aspects, the membrane includes a plurality of reagents. In some aspects, the membrane includes a functionalized surface. In various aspects, the reagent includes one or more of a nucleic acid, amino acid, protein, peptide, hormone, steroid, lipid, antigen, carbohydrate, ion or cofactor.

### DESCRIPTION OF THE FIGURES

FIGS. 1A-1D show BAF plots indicating that nuclear purification improves fetal DNA quality. The BAF frequency (B-allele frequency) plots shows comparisons of the genotypes of highly variable single nucleotide polymorphisms (Fetal cells to fetal placenta, fetal cells to maternal with and without nuclei isolation). Due to the genetic relationship between mother and fetus about 50% of the genotype is shared with the mother in a clean DNA isolates which is demonstrated in FIG. 1A. The placenta is similar to the fetal trophoblast cells isolated from the endocervical specimen and therefore the genotype should be similar (FIG. 1B). If the fetal sample is contaminated with maternal or other (e.g., paternal) DNA the BAF shifts towards the maternal (or e.g., paternal) genetic profile in a dose dependent manner If the contamination is too high the fetal DNA can be indistinguishable from the maternal genotype (FIG. 1C). As a result, the fetal sample will appear different from the placental DNA signature (FIG. 1D).

### DETAILED DESCRIPTION OF THE INVENTION

The present invention is based on the seminal discovery that fetal cells can be isolated from an endocervical sample from a pregnant subject. The invention further includes the isolation of target nucleic acid (e.g., fetal nucleic acid) from an endocervical sample containing target and non-target nucleic acid and the subsequent analysis of the target nucleic acid.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (e.g., greater than 50%) or essentially all of a device, substance, or composition.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

Described herein are methods, devices and kits for removal of non-target nucleic acid (e.g., maternal DNA) contamination from a sample using a combination of nuclear isolation with a solid matrix and the isolation of target nucleic acid. The recovery of target nucleic acid (fetal DNA) using the methods described herein is >80%, >85%, >90%, >95%, >96%, >97%, or >98% or >99%. The methods, devices and kits described herein enable fast DNA isolation suitable for commercial, high-throughput, automated processes with cell numbers as low as 1-10 cells, 10-25 cells, 25-50 cells, 50-100 cells, 100-250 cells, 250-500 cells, 500-750 cells, 750-1000 cells, 1000-2500 cells, and 2500-5000 cells. The methods, devices and kits described herein enable reliable diagnostic sample analysis, such as DNA isolation for sequencing, PCR, and whole genome amplification by providing efficient removal of non-target DNA.

While the present disclosure exemplifies use of nucleic acids for analysis, it will be understood that the methods, devices and kits of the invention allow for collection and analysis of any number of analytes to detect a condition, disease or disorder associated with the subject and/or fetus of the subject. As such, detection of the various conditions, diseases and disorders disclosed herein, is not limited to nucleic acid analysis.

In one embodiment, the present invention provides a method of isolating target nucleic acid from a sample of cells comprising incubating the cells from the sample on a DNA binding membrane or a DNA binding matrix with a protein cocktail containing at least one enzyme to free or release the cellular nuclei; washing the cells to remove non-target nucleic acid; lysing the nuclei to release the nucleic acid; and isolating the target nucleic acid. In one aspect the sample comprises non-target nucleic acid, maternal cells and/or fetal cells.

Biological samples can be contaminated with free floating nucleic acid that can influence the success of down-stream applications that focus on specific subpopulation of cells in a sample. Efficient removal of contaminating DNA (e.g., non-target DNA)is critical to obtain a high-quality readout for assays that are affected by such contaminations. The methods described herein combine the dislodging of cells and nuclear isolation by using enzymes or other comparable means (hypotonic solution) to bring all contaminants into solution. Nuclei are purified simply by adding nuclei and contaminants on a DNA binding matrix under non-binding conditions. Contaminants are washed through the column while nuclei will not pass. A simple change of pH or the use of chaotropic high salt solution will lyse the nuclei, release the DNA and bind it efficiently to any DNA binding matrix. This can be supported by an enzymatic digestion step. Organic solvents (Ethanol and others) can be used to remove salt and proteins efficiently from the bound DNA. DNA can than simply eluted from the column with any common elution buffer of choice that is compatible with the downstream application (e.g., H₂O, TRIS-HCL). During the DNA isolation process the cells are not fixed or bound to a surface (e.g., a membrane or matrix, either DNA binding or non-DNA binding).

The term “subject” as used herein refers to any individual or patient to which the subject methods are performed. Generally, the subject is human, although as will be appreciated by those in the art, the subject may be an animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject. In one aspect, the subject is a female human. In another aspect, the subject is pregnant. The pregnant subject maybe at least about 5 weeks, 6 weeks, 7, weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 25 weeks, 30 weeks, 35 weeks or 40 weeks of gestation.

The terms “nucleic acid” and “nucleic acid molecule” may be used interchangeably throughout the disclosure. The terms refer to a deoxyribonucleotide (DNA), ribonucleotide polymer (RNA), such as mRNA, tRNA and miRNA, RNA/DNA hybrids and polyamide nucleic acids (PNAs) in either single- or double-stranded form, and unless otherwise limited, would encompass known analogs of natural nucleotides that can function in a similar manner as naturally occurring nucleotides.

As used herein, the term “target nucleic acid” refers to the nucleic acid of interest that is extracted based on its cell of origin. In one aspect, the target nucleic acid is fetal nucleic acid.

As used herein, the term “non-target nucleic acid” refers to the non-desired background nucleic acid present in a biological sample. In one aspect, non-target nucleic acid is from a host or host cell. In another aspect, non-target nucleic acid is of maternal, viral or microbial origin or is cell free DNA. In one aspect, the protein cocktail used to free the cellular nuclei comprises pepsin. In another aspect, the protein cocktail does not comprise DNase. For example the cells are incubated in a pH7.5 buffer such as PBS or other and acidified with for example 1N HC1 to a final concentration of for example 0.04N or other that ensures pepsin activity.

Although less desirable if a sampling condition is chosen that does not allow free flow of ions in and out of the cells (e.g., live cells) a hypotonic solution could be used to free the nuclei from other cellular material. An exmaple hypotonic: 10 mM HEPES, pH 7.9, with 1.5 mM MgCl2 and 10 mM KCl and or any other buffer could be used that releases the nuclei into solution. Under certain circumstances the use of an isotonic buffer might be desirable for example; 10 mM Tris HCl, pH 7.5, with 2 mM MgCl2, 3 mM CaCl2, 0.3 M Sucrose.

In a further aspect, the nuclei are washed or incubated with a buffer resulting in that does not lyse the nuclei and allows other biological material being removed in the process. For example a membrane or matrix could be used to trap the nuclei while the wash buffer, for example PBS pH7.5 removes other biological material. Less desired but antibodies specific to nuclear envelope proteins could be used to immunodeplete the nuclei from the wash buffer.

For example, Anti-lamin A antibody can be coupled to a solid phase that enables isolation of nuclei from the complex solution.

Nuclei are then lysed with a proteinase for example proteinase K or trypsin and a buffer, for example a lysis buffer for example PBS pH 7.5 or any other that allows nuclei lysis and subsequent DNA isolation. In an additional aspect, the nucleic acid released following lysis binds to the membrane or matrix. In one aspect, the nucleic acid is isolated by elution from the DNA binding membrane or DNA binding matrix. In certain aspects, the lysis buffer is non-enzymatic.

The methods described herein relate to the extraction of nucleic acid from a biological sample such as whole blood, serum, plasma, umbilical cord blood, chorionic amniotic fluid, cerebrospinal fluid, spinal fluid, lavage fluid (e.g., bronchoalveolar, gastric, peritoneal, ductal, ear, arthroscopic), biopsy sample, urine, feces, sputum, saliva, nasal mucous, lymphatic fluid, bile, tears, sweat, breast milk, breast fluid, embryonic cells, fetal cells or an endocervical sample. As used herein, the term “endocervical sample” encompasses cells collected from the endocervical canal. In one aspect, the endocervical sample is from a pregnant subject. In another aspect, the endocervical sample contains non-target nucleic acid, maternal cells and/or fetal cells.

The method for nuclei isolation described herein includes: isolating a cell population; incubating the cell population with a digestion cocktail, such that the digestion cocktail removes all cell components besides the fetal nuclei and releases the foreign DNA into solution; applying the resulting digestion to a matrix under non-DNA binding conditions, such that the nuclei will be unable to pass through the matrix; applying a washing buffer to the matrix so that the foreign DNA and other cell components pass through the matrix; applying a nuclear lysis and DNA binding buffer to the matrix, such that the fetal nuclei are lysed, the matrix is in a DNA-binding state, and the fetal DNA binds to the matrix; washing the matrix with a wash buffer to remove unwanted chemicals and proteins; and eluting the fetal DNA using a buffer. The cells may be isolated or collected using a conventional menstrual cup or collection device of the invention. For the present invention, the isolated cells are not fixed or bound to a surface during target DNA isolation, e.g., fetal DNA. The surface can be a membrane or matrix, either DNA binding or non-DNA binding.

The term “extraction” as used herein refers to the partial or complete separation and isolation of a nucleic acid from a biological or non-biological sample comprising other nucleic acids. The terms “selective” and “selectively” as used herein refer to the ability to extract a particular species of nucleic acid molecule, on the basis of molecular size from a combination which includes or is a mixture of species of nucleic acid molecules.

The extraction of nucleic acid from biological material requires cell lysis, inactivation of cellular nucleases and separation of the desired nucleic acid from cellular debris. Common lysis procedures include mechanical disruption (e.g., grinding, hypotonic lysis), chemical treatment (e.g., detergent lysis, chaotropic agents, thiol reduction), and enzymatic digestion (e.g., proteinase K). The biological sample may be first lysed in the presence of a buffer, for example a lysis buffer, chaotropic agent (e.g., salt) and proteinase or protease. Cell membrane disruption and inactivation of intracellular nucleases may be combined. For instance, a single solution may contain detergents to solubilize cell membranes and strong chaotropic salts to inactivate intracellular enzymes. After cell lysis and nuclease inactivation, cellular debris may easily be removed by filtration or precipitation.

The method uses buffers or enzymes to set free nuclei from cells in solution on an inert mesh or matrix without the usage of DNase. For example, 1 to 10,000 target cells are exposed to enzymes or hypotonic solutions that result in the release of the cellular nuclei. The enzymes or hypotonic solution may be applied to the cells before they are added to the matrix or while the cells are on the matrix. In some embodiments, the matrix is configured to be able to bind DNA (e.g., silica matrices). The enzymes should preferably digest the cellular wall and not the nuclear envelope. An example of such an enzyme is pepsin. Following several washes with phosphate buffered saline or other buffers that do not induce DNA to matrix affinity or that may lyse the target cell nuclei.

For example, DNA can bind to various matrices such as silica under certain chemical conditions. Physiological buffers (PBS, TRIS) do not induce binding of DNA to a matrix nor lyse nuclei and allows unwanted DNA to pass through the column efficiently. Some of these buffers are used to elute bound DNA from matrices as shown below. In contrast, for example, guanidium HCl (GuHCl), which acts as a chaotrope results in nuclei lysis, DNA release, and activation of the silica matrix to bind DNA molecules tightly. Washes with, for example, high salt concentration or ethanol will not disrupt the binding and can be used to remove cellular material and salt. The clean DNA can then be eluted in water buffers, TE buffer, water, and others that reverse the binding capacity of the matrix to a non-DNA binding state resulting in DNA elution.

Lysis buffer (e.g., including Tris-HCl, EDTA, Triton X-100, NaCl, KCl, etc.) not exceeding the volume of the binding matrix in combination with a chaotropic salt or any other chemical that enables DNA matrix binding for purification purposes is added to the matrix which results in lysis of nuclei and its DNA release and activation of DNA binding to the matrix. The DNA binding matrix may be washed using organic solvent based washing buffers (e.g., acetic acid, acetone, acetonitrile, benzene, 1-butanol, 2-butanol, 2-butanone, t-butyl alcohol, carbon tetracholoride, chlorobenzene, chloroform, cyclohexane, 1,2-dichloroethane, diethylene glycol, diethyl ether, diglyme, DMA, DMF, DMSO, 1,4-dioxane, ethanol, ethyl acetate, ethylene glycol, glycerin, heptane, HMPA, HMPT, hexane, methanol, MTBE, methylene chloride, NMP, nitromethane, pentane, petroleum ether, 1-propanol, 2-popanol, pyridine, THF, toluene, triethyl amine, water, heavy water, o-xylene, m-xylene, p-xylene, etc.) to remove protein contaminants. Following lysis and after drying of the membrane, the DNA is eluted from the matrix under non-binding solvent conditions such as Tris EDTA or water. The lysis buffer can be an enzymatic or non-enzymatic lysis buffer.

The method may include adding a washing step or steps to remove non-nucleic acid molecules, for example salts, from the solid-support-target nucleic acid complex or surrounding solution. Non-nucleic acid molecules are then removed with an alcohol-based wash and the target nucleic acid is eluted under low- or no-salt conditions (TE buffer or water) in small volumes, ready for immediate use without further concentration. In another embodiment, extraction is improved by the introduction of a carrier such as tRNA, glycogen, polyA RNA, dextran blue, linear poly acrylamide (LPA), or any material that increases the recovery of nucleic acid. The carriers may be added to the second binding solution or washing buffer.

The methods described herein may be used in conjunction with any known technique suitable for the extraction, isolation or purification of nucleic acids, including, but not limited to, cesium chloride gradients, gradients, sucrose gradients, glucose gradients, centrifugation protocols, boiling, Microcon 100 filter, Chemagen viral DNA/RNA lk kit, Qiagen purification systems, Qiagen MinElute kits, HiSpeed Plasmid Maxi Kit, OlAfilter plasmid kit, Promega DNA purification systems, MangeSil Paramagnetic Particle based systems, Wizard SV technology, Wizard Genomic DNA purification kit, Amersham purification systems, Invitrogen Life Technologies Purification Systems, CONCERT purification system, and Mo Bio Laboratories purification systems.

In one aspect, the DNA binding membrane or DNA binding matrix is silica or other matrix material that under low pH and high salt binds DNA molecules. Typically, reducing the ionic strength and pH above 7.0 will result in DNA elution. A matrix pore size from <2 micron is preferred to ensure nuclei trapped in the matrix. DNA binding membrane or DNA binding matrix may refer to any surface having chemical and physical properties such that it is capable of adsorbing DNA. For instance, the electrostatic charge of a charged surface can be adjusted through pH change, which can render said surface more or less charged. With an appropriate buffer, when pH and salt concentration are optimal, the electrostatic charge of a surface can be modulated which can decrease the electrostatic repulsion between a negatively charged DNA and a negatively charged surface, or increase the electrostatic attraction between a negatively charged DNA and a positively charged surface; which favors the adsorption of the DNA to the surface. Any material capable of adsorbing negatively charged DNA might be used for this purpose. Silica is a non-limiting example of suitable material that can be used to form a DNA binding matrix. A non-DNA binding membrane to hold the nuclei could be used in an alternative embodiment, wherein the nuclei could be captured if the matrix pore size was too large.

Following isolation of the target nucleic acid the final relative percentage of target nucleic acid (e.g., fetal DNA) to non-target nucleic acid is at least about 5-6% fetal DNA, about 7-8% fetal DNA, about 9-10% fetal DNA, about 11-12% fetal DNA, about 13-14% fetal DNA. about 15-16% fetal DNA, about 16-17% fetal DNA, about 17-18% fetal DNA, about 18-19% fetal DNA, about 19-20% fetal DNA, about 20-21% fetal DNA, about 21-22% fetal DNA, about 22-23% fetal DNA, about 23-24% fetal DNA, about 24-25% fetal DNA, about 25-35% fetal DNA, about 35-45% fetal DNA, about 45-55% fetal DNA, about 55-65% fetal DNA, about 65-75% fetal DNA, about 75-85% fetal DNA, about 85-90% fetal DNA, about 90-91% fetal DNA, about 91-92% fetal DNA, about 92-93% fetal DNA, about 93-94% fetal DNA, about 94-95% fetal DNA, about 95-96% fetal DNA, about 96-97% fetal DNA, about 97-98% fetal DNA, about 98-99% fetal DNA, or about 99-99.7% fetal DNA.

If DNA quantification post purification does not match the expected DNA amount, loss of DNA in the purification process can be assumed. To reduce this loss, artificial DNA or RNA (depending on the downstream application) may be used in empirically established amounts to increase target DNA binding to the matrix and its recovery.

In another aspect, the non-target nucleic acid contamination is less than about 1%, 2%, 3%, 4%, 5%, 10%, 15%, 20% or less than about 50%. In a further aspect, the isolated target nucleic acid is analyzed by DNA sequencing, PCR or whole genome amplification.

Fetal cells can be isolated from the cervical canal using immunodepletion techniques. In general, fetal cells are present in ratios from 1 in 2000 to 1 in 10,000 maternal cells. Using fetal cell specific antibodies, fetal cells are enriched near to purity by leaving maternal cells behind. Although fetal cells are nearly pure shown by FISH analysis for a male baby, the detection analysis of the fetal DNA can be masked by the overwhelming amount of non-target (e.g., maternal DNA present in the sample), indicating that non-target DNA is present extracellularly and/or in the fetal cell.

To allow precise DNA analysis, a high-quality DNA sample is needed. The amount of target DNA and the amount of contamination are directly proportional to the success of analysis such as sequencing and PCR with and without whole genome amplification. Lower cell numbers of the target cells require increased consistency of non-target DNA removal and target DNA recovery. Results, after using the kits and methods described herein, show high purity (e.g., >50% >80%, >85%, >90%, >85%, >98%) with as little as 1-25, 25-50, 50-100, 100-250, 250-500, 500-750, 750-1000, 1000-2000, or 2000-5000 target cells in the sample.

There are a variety of non-invasive and invasive techniques available for prenatal diagnosis including ultrasonography, amniocentesis, chorionic villi sampling (CVS), fetal blood cells in maternal blood, maternal serum alpha-fetoprotein, maternal serum beta-HCG, and maternal serum estriol. However, the techniques that are non-invasive are less specific, and the techniques with high specificity and high sensitivity are highly invasive. Furthermore, most techniques can be applied only during specific time periods during pregnancy for greatest utility.

The first marker that was developed for fetal DNA detection in maternal plasma was the Y chromosome, which is present in male fetuses. The robustness of Y chromosomal markers has been reproduced by many workers in the field. This approach constitutes a highly accurate method for the determination of fetal gender, which is useful for the prenatal investigation of sex-linked diseases. Maternal plasma DNA analysis is also useful for the noninvasive prenatal determination of fetal RhD blood group status in RhD-negative pregnant women. More recently, maternal plasma DNA analysis has been shown to be useful for the noninvasive prenatal exclusion of fetal β-thalassemia major. A similar approach has also been used for prenatal detection of the HbE gene.

Other genetic applications of fetal DNA in maternal plasma include the detection of achondroplasia, myotonic dystrophy, cystic fibrosis, Huntington disease, and congenital adrenal hyperplasia. It is expected that the spectrum of such applications will increase over the next few years.

For the methods described herein, the subject is pregnant and the method of evaluating a disease or physiological condition in the subject or her fetus aids in the detection, monitoring, prognosis or treatment of the subject or her fetus. More specifically, the present invention features methods of detecting abnormalities in a fetus by detecting fetal DNA in a biological sample obtained from a mother. The methods according to the present invention provide for detecting fetal DNA in a maternal sample by differentiating the fetal DNA from the maternal DNA. Employing such methods, fetal DNA that is predictive of a genetic anomaly or genetic-based disease may be identified thereby providing methods for prenatal diagnosis. These methods are applicable to any and all pregnancy-associated conditions for which nucleic acid changes, mutations or other characteristics (e.g., methylation state) are associated with a disease state. For example, sequence analysis can be used to detect single nucleotide polymorphisms (SNPs) and DNA mutations such as insertions and/or deletions. Exemplary diseases that may be diagnosed include, for example, preeclampsia, preterm labor, hyperemesis gravidarum, ectopic pregnancy, fetal chromosomal aneuploidy (such as trisomy 18, 21, or 13), and intrauterine growth retardation.

The methods, devices and kits described herein allow for the analysis of fetal genetic traits including those involved in chromosomal aberrations (e.g., aneuploidies or chromosomal aberrations associated with Down's syndrome) or hereditary Mendelian genetic disorders and, respectively, genetic markers associated therewith (e.g., single gene disorders such as cystic fibrosis or the hemoglobinopathies).

In an additional embodiment, the present invention provides a method of analyzing fetal nucleic acid from an endocervical sample comprising; isolating fetal cells from the endocervical sample; incubating the fetal cells on a DNA binding membrane or a DNA binding matrix with a protein cocktail comprises an enzyme to free or release the cellular nuclei; washing the fetal nuclei to remove non-target nucleic acid; lysing the nuclei to release the fetal nucleic acid; and isolating the fetal nucleic acid. In one aspect, the endocervical sample is collected using a menstrual cup. In an additional aspect, the endocervical sample comprises non-target nucleic acid, maternal cells and/or fetal cells. In a further aspect, the protein cocktail comprises a proteinase that preferentially digests the cellular wall and not the nuclear envelope. In certain aspects, the nuclei are lysis using an enzymatic or non-enzymatic lysis buffer. The lysis buffer may comprise proteinase K and/or trypsin.

In general, fetal cells are present in ratios from 1 in 2000 to 1 in 10,000 maternal cells. Using fetal cell specific antibodies, fetal cells are enriched near to purity by leaving maternal cells behind. In the methods described herein, the fetal cells are isolated by binding of the cells to anti-HLA-G. HLA-G histocompatibility antigen, class I, G, also known as human leukocyte antigen G (HLA-G), is a protein that in humans is encoded by the HLA-G gene. HLA-G belongs to the HLA nonclassical class I heavy chain paralogues. This class I molecule is a heterodimer consisting of a heavy chain and a light chain (beta-2 microglobulin). HLA-G is expressed by fetal cells. In one aspect, the fetal cells are isolated using anti-HLA-G antibody coated nanoparticles. In some embodiments, the fetal cells are analyzed by flow cytometry, immunostaining, microscopy, polymerase chain reaction, sequencing, or any other methods known to one of skill in the art.

In an additional aspect, the sample comprises about 1-10 cells, 10-25 cells, 25-50 cells, 50-100 cells, 100-250 cells, 25-500 cells, 500-750 cells, 750-1000 cells, 1000-2500 cells or 2500-5000 cells. In an aspect, the protein cocktail used to free cellular nuclei comprises at least one enzyme, which preferentially digests the cellular wall and not the nuclear envelope. An example of such an enzyme is pepsin. In a further aspect, the protein cocktail preferably does not contain DNase. In some aspects, the nuclei are lysed by incubating the cells with a lysis buffer. The lysis buffer can be enzymatic or non-enzymatic. The lysis buffer may comprise proteinase K and/or trypsin. In another aspect, the released fetal nucleic acid binds to the membrane or matrix. In a further aspect, the fetal nucleic acid is isolated by eluting the nucleic acid from the membrane. In one aspect, non-target nucleic acid contamination is less than about 1%, 2%, 3%, 4%, 5%, 10%, 15%, 20% or less than about 50%.

The term “pregnancy-associated disorder,” as used herein, refers to any condition or disease that may affect a pregnant woman, the fetus the woman is carrying, or both the woman and the fetus. Such a condition or disease may manifest its symptoms during a limited time period, e.g., during pregnancy or delivery, or may last the entire life span of the fetus following its birth. Some examples of a pregnancy-associated disorder include ectopic pregnancy, preeclampsia, preterm labor, and fetal chromosomal abnormalities such as trisomy 13, 18, or 21.

The term “chromosomal abnormality” refers to a deviation between the structure of the subject chromosome and a normal homologous chromosome. The term “normal” refers to the predominate karyotype or banding pattern found in healthy individuals of a particular species. A chromosomal abnormality can be numerical or structural, and includes but is not limited to aneuploidy, polyploidy, inversion, a trisomy, a monosomy, duplication, deletion, deletion of a part of a chromosome, addition, addition of a part of chromosome, insertion, a fragment of a chromosome, a region of a chromosome, chromosomal rearrangement, and translocation. A chromosomal abnormality can be correlated with presence of a pathological condition or with a predisposition to develop a pathological condition.

Examples of fetal diseases or conditions resulting from genetic anomalies, gene mutations and chromosomal abnormalities include achondroplasia, Down syndrome, trisomy 21, trisomy 18, trisomy 13, Turner syndrome, Sickle cell disease, Cystic fibrosis, fragile XD syndrome, Muscular dystrophy (e.g., Duchenne muscular dystrophy), Tay-Sachs disease, Neural tube defects, such as spina bifida and anencephaly, Thalassemia, Polycystic kidney disease, Hemophilia A, Huntington's disease and congenital adrenal hyperplasia.

Described herein are devices, kits and methods for collecting an endocervical sample. The methods described herein may use a collection device of the present invention or a foldable cup such as a menstrual cup, a storage container and a transport solution to enable safe ‘at home sampling’ of cervical cells originating from the cervical canal between 5 and 20 weeks of pregnancy. The kits described herein may be safely used by both healthcare professionals as well as individuals at home.

A menstrual cup as described herein is a funnel shaped reusable device that is placed by the woman just below the cervical canal. The cup may be configured in various sizes and shapes to ensure comfort and maximum collection of cells. The cup is positioned under the opening of the cervical canal after confirmed pregnancy. The cup can also be positioned under the opening of the cervical canal before confirmed pregnancy to, for example, collect cells or a sample that may be used in identifying or confirming pregnancy. The cup is inserted at the opening of the cervical canal as for as long as at least one fetal cell is collected by the cup. For example, the cup may be positioned at the opening of the cervical canal for up to one hour, five hours, ten hours, twelve hours, fifteen hours, twenty hours, or any range or subrange there between. The cup is advantageous in that it is a non-invasive technique to collect cells compared to other methods such as a Pap smear.

In a related embodiment, an endocervical sample may be collected using a collection device of the present invention. The device is designed to be disposable, e.g., intended for single use and includes a ring having a central opening, and a membrane coupled to the ring covering the central opening. The device may be configured as a disposable resiliently deformable and flexible cup or disc which is suitable for being disposed at a location proximal to an external os of a uterine cervix or anywhere within the vaginal canal of the subject to collect an endocervical sample. The ring of the device forms the rim of the collection device while the membrane forms a reservoir for collecting sample. The ring may be sized to fit directly and/or snugly over the external os of the cervix or sized to be disposed at a location adjacent the external os of the cervix, for example just below the cervical canal, such that endocervical fluid is collected in the reservoir. The ring and membrane are composed of a resiliently deformable material with the ring being generally circular or oval in a non-deformed configuration. The resilient nature of the device allows for the device to be compressed for ease of placement of the device proximal to the cervical canal or anywhere within the vaginal canal and subsequent removal of the device by a subject. In various embodiments, the device may be positioned proximal to the cervical canal or anywhere within the vaginal canal for up to one hour, five hours, ten hours, twelve hours, fifteen hours, twenty hours, or any range or subrange there between. In some embodiments, the device is sized to collect at least about 0.5, 1, 2, 3, 4 or 5 mL of endocervical sample. In some embodiments, the device is sized to collect less than about 0.5, 1, 2, 3, 4 or 5 mL of endocervical sample.

The collection device of the invention is resiliently deformable and composed of one or more resiliently deformable biocompatible materials so long as the materials are substantially non-degradable. Such materials include biocompatible polymers. Biocompatible materials for use in the collection device include, but are not limited to polyethylene (PE), nylon, polyamide, polyether block amides (PEBAX), polyethylene terephthalate (PET), silicone, POC, polypropylene and polyether block PBT. In one embodiment, the entire device is composed of the same material. In another embodiment, the ring and membrane are formed of different materials.

In embodiments, the membrane of the collection device may have a reagent for performing an assay deposited thereon. Any number of reagents are known in the art and envisioned for use with the present invention include, for example, one or more of a nucleic acid, amino acid, protein, peptide, hormone, steroid, lipid, antigen, carbohydrate, ion or cofactor. Inclusion of a reagent allows for an assay to be performed upon contact with a sample, such as immobilization of an analyte.

In further embodiments, the membrane of the collection device may be functionalized A surface may be referred to as “functionalized” when it includes a linker, a scaffold, a building block, or other reactive moiety attached thereto, whereas a surface may be “nonfunctionalized” when it lacks such a reactive moiety attached thereto.

A functionalized membrane may refer to the surface of the membrane comprising a functional group. A functional group may be a group capable of forming an attachment with another functional group. For example, a functional group may be biotin, which may form an attachment with streptavidin, another functional group. Exemplary functional groups may include, but are not limited to, aldehydes, ketones, carboxy groups, amino groups, biotin, streptavidin, nucleic acids, small molecules (e.g., for click chemistry), homo- and hetero-bifunctional reagents (e.g., N-succinimidyl(4-iodoacetyl) aminobenzoate (STAB), dimaleimide, dithio-bis-nitrobenzoic acid (DTNB), N-succinimidyl-S-acetyl-thioacetate (SATA), N-succinimidyl-3-(2-pyridyldithio) propionate (SPDP), succinimidyl 4-(N-mafemimdomethyl)-cyclohexame-1-carboxylate (SMCC) and 6-hydrazinonicotimide (HYNIC), and antibodies. In some instances, the functional group is a carboxy group (e.g., COOH).

The membrane surface may be treated or coated to render the surface hydrophilic or hydrophobic. A variety of surface treatments and surface modification techniques may be used to alter the properties of the membrane surface. Examples include, but are not limited to, oxygen plasma treatments to render hydrophobic material surfaces more hydrophilic, the use of wet or dry etching techniques to smooth (or roughen) silicon surfaces, adsorption and/or grafting of polyethylene oxide or other polymer layers to substrate surfaces to render them more hydrophilic and less prone to non-specific adsorption of biomolecules and cells, the use of silane reactions to graft chemically-reactive functional groups to otherwise inert silicon surfaces. Photodeprotection techniques can be used to selectively activate chemically-reactive functional groups at specific locations of the membrane, for example, the selective addition or activation of chemically-reactive functional groups such as primary amines or carboxyl groups on the membrane surface may be used to covalently couple oligonucleotide probes, peptides, proteins, or other biomolecules to the membrane surface. In general, the choice of surface treatment or surface modification utilized will depend both on the type of surface property that is desired and on the type of material from which the substrate is made.

The cup or device of the invention is carefully removed and placed into a storage container that is or will be filled with transport media for shipment and subsequent analysis. For example, the transport media may include one or more cell preservation chemicals (e.g., glycerol, serum, dimethyl sulfoxide, methanol, acetic acid, cell culture medium, a desiccation agent, etc.). The sample may also be collected without using a transport medium and sealed in the storage container.

In a further embodiment, the present invention provides a kit for the collection of an endocervical sample comprising a foldable menstruation cup or collection device of the invention; a storage container; and with or without transport media. In one aspect, the collection device or menstruation cup is inserted into the vaginal canal. In another aspect, the collection device or menstruation cup is inserted for a time and under conditions to allow for sample collection, for example, for about 10 minutes, 15, minutes, 20 minutes, 25 minutes, 30 minutes, 35 minutes, 40 minutes, 45 minutes, 50 minutes, 55 minutes, 60 minutes, less than one hour, 1-2 hours, 1-5 hours, 1-10 hours, 1-20 or more hours. In a further aspect, the transport media comprises at least one cell preservation chemical. In certain aspects, the preservation chemical is glycerol, serum, dimethyl sulfoxide, methanol, acetic acid, cell culture medium and/o a desiccation agent.

In a further embodiment, the invention provides a method for conducting an assay utilizing the collection device of the invention. The method includes conducting an assay to detect an analyte present in an endocervical sample from a subject, wherein the sample is collected into a disposable resiliently deformable and flexible cup or disc of the invention disposed at a location proximal to an external os of a uterine cervix or anywhere within the vaginal canal of the subject. In embodiments, the method further includes isolating the analyte from the endocervical sample and optionally analyzing the analyte.

In still another embodiment, the invention provides a method of collecting an endocervical sample and analyzing the sample. The method includes: a) disposing a disposable resiliently deformable and flexible cup or disc of the invention at a location proximal to an external os of a uterine cervix or anywhere within the vaginal canal of a subject; b) collecting an endocervical sample into the disposable resiliently deformable and flexible cup or disc while the cup or disc is located proximal to the external os or anywhere within the vaginal canal; c) removing the cup or disc containing the endocervical sample from the subject; and d) conducting an assay to detect an analyte present in the endocervical sample.

The methods and devices of the invention allow a subject to easily collect an endocervical sample in a non-clinical setting, e.g., an at-home setting, which can be subsequently analyzed in a clinical setting. For example, once the sample is collected by the subject in an at-home setting, the sample is transported to a clinical setting for diagnostic analysis.

While the present disclosure exemplifies use of nucleic acids for analysis, it will be understood that the methods of the invention include analysis of any number of analytes to detect a disease or condition associated with the subject and/or fetus of the subject. For example, an analyte may include, but not limited to, a cell or portion thereof, cellular nuclei or portion thereof, a biomolecule, such as a nucleic acid (e.g., DNA, RNA, mRNA, tRNA or miRNA), amino acid, protein, peptide, hormone, steroid, lipid, carbohydrate or ion, a chemical compound, such as a small organic compound, a microorganism or portion thereof (e.g., virus, bacteria or fungi). In one aspect, the analyte is human chorionic gonadotropin. Given the number of potential analyte targets, it is possible to detect a myriad of different conditions, diseases and/or disorders.

In certain aspects, analysis includes detecting a disease or condition associated with the subject, including for example, cancer, sexually transmitted infection, pregnancy, fetal defect, status of microbiome or presence of a microorganism.

In certain aspects, analysis includes detecting a marker for female reproductive management, such as miscarriage, fetal growth restriction, preterm labor, ruptured membrane, or any combination thereof.

In certain aspects, analysis includes detecting a marker for female reproductive oncology, such as cancer, including but not limited to ovarian, uterine, or cervical cancer.

In certain aspects, analysis includes detecting a marker of a female reproductive disorder, such as endometriosis or polycystic ovarian syndrome.

In certain aspects, analysis includes detection of an infection, such as a uterine infection.

In certain aspects, analysis includes detecting the presence and strain type of a virus, such as human papillomavirus.

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLES

### Example 1

### Isolation of Target DNA From a Mixed Sample

One to 2,000 female cells were incubated with and without 2 to 10,000 fold of male standard DNA for one hour in culture media. Ten-fold fixative was added to mimic DNA sticking to target cells. Cells where harvested, counted, and exposed to a protein cocktail that freed nuclei. The mixture was aliquoted onto a matrix (e.g., a matrix within a column). Control cells were used without the nuclei release procedure.

The columns were spun for ten seconds at 8000×g in a microcentrifuge. Five hundred microliters of phosphate buffered saline was added twice to the matrix to wash out contaminating DNA. Lysis binding buffer was added to initiate nuclei DNA release and matrix binding. Samples below fifty cells received carrier RNA spiked into the lysis buffer to ensure efficient binding.

DNA was washed with an Ethanol based solution and the matrix was dried thereafter, before eluting the DNA using ten microliters of TRIS EDTA pH 8 buffer.

DNA was quantified using quantitative PCR for RNaseH (total copy number) and sex-determining region Y (SRY). Results showed >90% removal of non-target DNA.

### Example XAMPLE 2

### Cervical Sample Collection

At fourteen weeks of pregnancy a volunteer used a menstrual cup for twelve hours over night providing a total of 25 million cells. About 250 fetal cells were identified by immunostaining for fetal HLAG and βhCG. Another six samples were collected for twelve hours (one was for six hours) from four patients using a menstrual disc (Flex), providing an average of 7 million cells. An average of 158 fetal cells were isolated by immunodepletion (range=60 to 250 cells), with an average of 93% (SD=1.6%) of the isolated cells identified as fetal by immunostaining for βhCG.

### Example 3

### TRIC-Fetal Cell Isolation Protocol from an Endocervical Specimen

Preparation of nanoparticles with bound anti-HLA-G. One day before the procedure, magnetic nanoparticles coupled to goat anti-mouse IgG (Clemente and Assoc.) were incubated with mouse monoclonal anti-HLA-G antibody (BD). 100 μL sterile PBS, 10 μL antibody (0.5 mg/mL), and 10 μL nanoparticles were combined and incubated overnight with mixing on the rocker in cold room at 4° C. The next day, unbound antibody was removed by first adding 900 μL sterile PBS and then magnetizing the particles 10 min before removing all liquid. The tip of a 200 μL Pipetman was placed against the opposite wall of the tube, and the liquid was drawn out slowly while moving the tip to the bottom of the tube. The particles were resupended in 1 mL sterile PBS and washed 2 more times, with 100 μL added for the final resuspension.

The initial endocervical sample arrived in 10 mL of Cytolyt solution. Using a plastic spatula, clumps of cells floating in the solution were broken up, and any visible material was scraped from the cytobrush, if left in the vial. (Optional: Add 500 μL of undiluted acetic acid with mixing to break up excess mucus, if it is problematic.) 10 μL of the cell suspension was aliqotted onto the hemocytometer and count cells and the total cells in the starting material was counted.

100 μL of the sample was removed and spun onto a microscope slide, using Shandon Cytospin. Use Shandon EZ Megafunnel disposable sample chamber. Initial cell sample was stained with anti-HLA-G mouse monoclonal antibody (BD #557577) and DAB. Cells were counterstained with hematoxylin, and counted to get an estimate of the HLA-G positive cell number (total on slide×20=total in sample). The ratio of trophoblasts/total cells can be calculated (total #HLA-G positive cells on slide×20/total # cells determined from hemocytometer count). The ratio should be about 1:2000.

Cells were pelleted at 1200 rpm (400×g) for 5 minutes (removes of all the fixative). The cell pellet was resuspended in 12-13 mL sterile PBS to a final volume of 14 mL. Optionally, pass the sample through a 250 μm tissue strainer inserted into a 15 mL centrifuge tube to remove large pieces of mucus and cell clumps.

The cervical sample was washed 2 times by centrifugation and resuspension of cells in 14 mL sterile PBS.

The sample was resuspended in 1.4 mL of sterile PBS. The entire 100 μL of anti-HLA-G-coated magnetic nanoparticles (prepared in #1) was added to the sample to isolate trophoblast cells and the sample was incubated overnight on the rocker in cold room at 4° C.

After the overnight incubation, the trophoblast cells were separated on the magnet (DynaMag-Spin magnet; Life Technologies) for 10 minutes at 4° C. Unbound maternal cells were removed by pipetting against the opposite wall of the tube, and drawing out the liquid slowly while moving the tip to the bottom of the tube. The trophoblast cells were washed 3 times with 1 mL sterile PBS at 4° C., using the magnet (allow nano particles to magnetize 10 minutes for each wash before pipetting). After the final removal of unbound cells, The captured cells were resuspended in 100 μL of sterile PBS at 4° C.

A small aliquot of the isolated cell suspension (10 μL) was removed and the isolated fetal cells were counted to calculate the total number of fetal cells recovered. The maternal cells from the first wash were counted, using the hemocytometer.

Cells were prepared for DNA isolation by treating the trophoblast cells with or w/o DNase immobilized on beads.

Slides were prepared for purity analysis, protein marker staining and FISH analysis. Approximately 50-100 cells spun onto each slide with cytospin, then heat the slide for 1 minute. ***Alternatively, place 40-100+ cells in a small drop (˜10-40 μL) in center of slide, heat on slide warmer to 45° C. for 5-10 min until dry.

The purity of the cells was determined by immunofluorescently labeling cells with anti-βhCG. The number of fluorescent βhCG positive cells and total cells (DAPI labeled) was determined and the % βhCG positive cells was found to be greater than 85%

### Example 4

### Fetal DNA Isolation

20× Pepsin was prepared (0.22g of pepsin in 50 ml 0.01N HCl). 100 μl of 20× pepsin was added to 100 μl of TRIC cells (isolated as described above) and incubated for 11 minutes at 37° C. on Eppendorf ThermoMixer C (500 rpm).

The cells were passed through a spin column (DNeasy blood & tissue), spin at 600 g for 30 sec and washed 5× with 500 μl of PBS by spinning at 600 g for 30 sec.

200 ul of PBS, 20 ul Proteinase K, 200 ul AL lysis buffer (DNeasy blood & tissue) were added to the column and the column was placed the column on Eppendorf ThermoMixer C (500 rpm) for 10 mins at 56° C.

200 ul of ETOH was added to the above mix [PBS+Proteinase K+AL buffer] and mixed by pipetting up and down and then spin at 6000 g for 1 min

The DNeasy Mini spin column was placed in a new 2 mL collection tube, 500 μL Buffer AW1 was added, and centrifuged for one minute at 6000×g (8000 rpm). The flow-through and collection tube were discarded.

The DNeasy Mini spin column was placed in a new 2 mL collection tube, 500 μL Buffer AW2 was added, and centrifuges for 3 minutes at 20,000×g (14,000 rpm) to dry the DNeasy membrane. The flow-through and collection tube were discarded.

It is important to dry the membrane of the DNeasy spin column, since residual ethanol may interfere with subsequent reactions. This centrifugation step ensures that no residual ethanol will be carried over during the following elution.

The DNeasy Mini spin column was placed in a clean 1.5 or 2 mL microcentrifuge tube, and 25 μL Buffer AE was pipetted directly onto the DNeasy membrane and incubated at room temperature for one minute, and then centrifuged for one minute at 6000×g (8000 rpm) to elute. The incubation and centrifugation elution step were repeated by adding the 25 μL Buffer AE from the first elution to the membrane. Store at −20° C. all-purity, amount of DNA obtained, data to show even with contamination (some) you have DNA that can be analyzed (so sequence data could help but not sure it's critical). The more data we put into the application, the better. Now is the time to add as much as possible.

### Example 5

### Analysis of DNA Isolated From Fetal Cells

DNA isolation from fetal cells obtained endocervical specimen contaminated with foreign/maternal DNA. Trophoblast cells (260-380 cells) were isolated from endocervical specimen (Samples A-D) with purities greater than 90% determined by immune-histochemistry (hCG positive). Fetal cells were split and processed for DNA was extraction with/without nuclei isolation. The DNA was isolated and analyzed by next generation sequencing using a method similar to the Illumina Forenseq technology. Highly variable identity SNPs were used to create specific genetic signatures from mother and fetus using reference DNA. The data was used to determine fetal and maternal DNA content in the isolated fetal cells from the endocervical specimens. The fetal fraction without nuclei isolation resulted in low fetal fraction below 11.5% and maternal contamination of 88.5-90.5% in this subset of samples (Table 1). Nuclei isolation reduced the maternal contamination to 2-10% and maternal and a high fetal fraction and purity of 90-98% (Table 1).

TABLE 1

| | | | Median | | Median | |
|---|---|---|---|---|---|---|
| | | Fetal | maternal | Fetal | maternal | Fetal |
| | No. of fetal | cell | contamination | Fraction | contamination | Fraction |
| | trophoblast | purity | (%) | (%) | (%) | (%) |
| Sample ID | cells | (%) | Nuclei isolation | | No nuclei isolation | |
| Sample A | 380 | 93.9 | 2 | 98 | 90.5 | 9.5 |
| Sample B | 260 | 92 | 5 | 95 | 90 | 10 |
| Sample C | 290 | 93 | 10 | 90 | 92 | 8 |
| Sample D | 340 | 91 | 5 | 95 | 88.5 | 11.5 |

### Example 6

### Analysis of Maternal DNA Contamination in Fetal DNA Sample

DNA isolation was performed post nuclei isolation from fetal cells obtained from endocervical specimen contaminated with foreign/maternal DNA as described previously. Trophoblast cells were initially isolated from endocervical specimen by immunodepletion (Sample 1-30). The DNA was isolated and analyzed by next generation sequencing using a method similar to the Illumina Forenseq technology. Highly variable identity SNPs were used to create specific genetic signatures from mother and fetus using reference DNA. The data was used to determine fetal and maternal DNA content in the isolated fetal cells from the endocervical specimens. Nuclei isolation reduced the maternal contamination to 0.5-16.7% and maternal and a high fetal fraction and purity of 83.3-99.5% (Table 2).

TABLE 2

| | | Median | |
|---|---|---|---|
| | No. of | maternal | Fetal |
| Sample | fetal | contamination | Fraction |
| ID's | cells | (%) | (%) |
| Sample 1 | 110 | 4.4 | 95.6 |
| Sample 2 | 180 | 9.7 | 90.3 |
| Sample 3 | 70 | 6.2 | 93.8 |
| Sample 4 | 440 | 13.1 | 86.9 |
| Sample 5 | 450 | 12.1 | 87.9 |
| Sample 6 | 340 | 3.2 | 96.8 |
| Sample 7 | 290 | 5.6 | 94.4 |
| Sample 8 | 290 | 3.2 | 96.8 |
| Sample 9 | 290 | 8.2 | 91.8 |
| Sample 10 | 310 | 5.1 | 94.9 |
| Sample 11 | 150 | 16.2 | 83.8 |
| Sample 12 | 260 | 16.7 | 83.3 |
| Sample 13 | 320 | 11.9 | 88.1 |
| Sample 14 | 200 | 14.8 | 85.2 |
| Sample 15 | 140 | 13.2 | 86.8 |
| Sample 16 | 180 | 10.5 | 89.5 |
| Sample 17 | 380 | 1.7 | 98.3 |
| Sample 18 | 210 | 12.3 | 87.7 |
| Sample 19 | 260 | 12.4 | 87.6 |
| Sample 20 | 260 | 0.5 | 99.5 |
| Sample 21 | 310 | 14.8 | 85.2 |
| Sample 22 | 160 | 15.5 | 84.5 |
| Sample 23 | 400 | 12.2 | 87.8 |
| Sample 24 | 180 | 12.1 | 87.9 |
| Sample 25 | 110 | 11.4 | 88.6 |
| Sample 26 | 110 | 16.7 | 83.3 |
| Sample 27 | 390 | 20 | 80 |
| Sample 28 | 160 | 13.7 | 86.3 |
| Sample 29 | 110 | 14.4 | 85.6 |
| Sample 30 | 110 | 10.8 | 89.2 |

### Example 7

### Analysis of Fetal DNA With and Without Nuclei Isolation

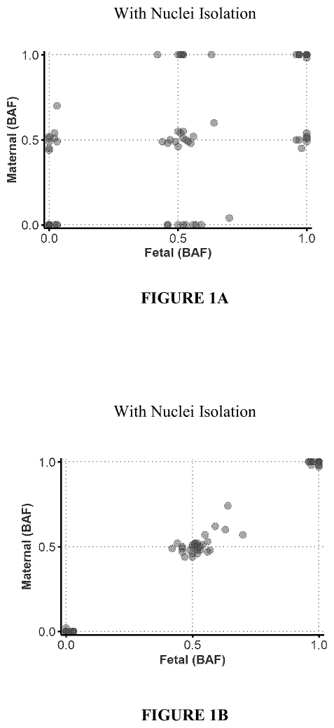

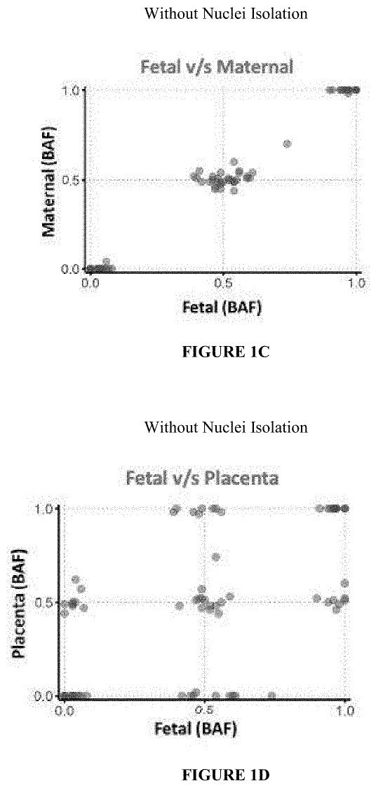

Fetal DNA isolation was performed pre and post nuclei isolation from fetal cells obtained from endocervical specimen contaminated with foreign/maternal DNA as described previously. Nuclei isolation prior to DNA isolation improved fetal DNA quality (FIG. 1 and Table 3). The correlation with nuclear isolation reached nearly 1 (0.98). Without nuclear isolation the fetal sample highly correlated with the mother due to unsuccessful removal of maternal DNA.

TABLE 3

| | With | Without |
|---|---|---|
| DNA extraction post | Nuclei | Nuclei |
| Nuclei isolation | isolation | isolation |
| Number of cells used | 190 | 190 |
| Relatedness score with | 0.49 | 0.98 |
| Maternal (expected ~0.5) | | |
| Relatedness score with | 0.98 | 0.47 |
| Placenta (expected >0.9) | | |

Although the invention has been described with reference to the above examples, it will be understood that modifications and variations are encompassed within the spirit and scope of the invention. Accordingly, the invention is limited only by the following claims.

1. A method comprising: conducting an assay to detect an analyte present in an endocervical sample from a subject, wherein the sample is collected into a disposable resiliently deformable and flexible cup or disc disposed at a location proximal to an external os of a uterine cervix or anywhere within the vaginal canal of the subject.

2. The method of claim 1, further comprising isolating the analyte from the endocervical sample.

3. The method of claim 1, wherein the analyte is a cell or portion thereof, or cellular nuclei or portion thereof.

4. The method of claim 1, wherein the analyte is a biomolecule selected from the group consisting of a nucleic acid, amino acid, protein, peptide, hormone, steroid, lipid, carbohydrate and ion.

5. The method of claim 1, wherein the analyte is a nucleic acid selected from the group consisting of DNA and RNA.

6. The method of claim 5, wherein the RNA is selected from the group consisting of mRNA, tRNA and miRNA.

7. The method of claim 1, wherein the analyte is a chemical compound.

8. The method of claim 1, wherein the analyte is a microorganism or portion thereof.

9. The method of claim 8, wherein the microorganism is a virus, bacteria or fungi.

10. The method of claim 1, further comprising analyzing the analyte.

11. The method of claim 10, wherein analysis comprises detecting a disease or condition.

12. The method of claim 11, wherein the disease or condition is selected from the group consisting of cancer, sexually transmitted infection, pregnancy, fetal defect, status of microbiome and presence of a microorganism.

13. The method of claim 10, wherein analysis comprises detecting a marker for female reproductive management.

14. The method of claim 13, wherein the marker is miscarriage, fetal growth restriction, preterm labor, ruptured membrane, or any combination thereof.

15. The method of claim 10, wherein analysis comprises detecting a marker of female reproductive oncology.

16. The method of claim 15, wherein the marker is of cancer.

17. The method of claim 16, wherein the cancer is ovarian, uterine, or cervical cancer.

18. The method of claim 10, wherein analysis comprises detecting a marker of a female reproductive disorder.

19. The method of claim 18, wherein the disorder is endometriosis or polycystic ovarian syndrome.

20. The method of claim 10, wherein analysis comprises detecting infection.

21. The method of claim 20, wherein the infection is uterine infection.

22. The method of claim 11, wherein analysis comprises determining the presence and strain type of a virus.

23. The method of claim 13, wherein the virus is human papillomavirus.

24. The method of claim 1, wherein the analyte is human chorionic gonadotropin.

25. The method of claim 1, wherein the disposable resiliently deformable and flexible cup or disc further comprises a reagent for performing an assay.

26. A method comprising:
a) disposing a disposable resiliently deformable and flexible cup or disc at a location proximal to an external os of a uterine cervix or anywhere within the vaginal canal of a subject;
b) collecting an endocervical sample into the disposable resiliently deformable and flexible cup or disc while the cup or disc is located proximal to the external os or anywhere within the vaginal canal;
c) removing the cup or disc containing the endocervical sample from the subject; and
d) conducting an assay to detect an analyte present in the endocervical sample, wherein (a)-(c) are performed by the subject.

27. The method of claim 26, wherein (a)-(d) are performed by the subject.

28. The method of claim 26, wherein (a)-(c) are performed in an at-home setting and (d) is performed by a clinician other than the subject in a clinical setting.

29. The method of claim 26, further comprising isolating the analyte from the endocervical sample.

30. The method of claim 26, wherein the analyte is a cell or portion thereof, or cellular nuclei or portion thereof.

31. The method of claim 26, wherein the analyte is a biomolecule selected from the group consisting of a nucleic acid, amino acid, protein, peptide, hormone, steroid, lipid, carbohydrate and ion.

32. The method of claim 26, wherein the analyte is a nucleic acid selected from the group consisting of DNA and RNA.

33. The method of claim 32, wherein the RNA is selected from the group consisting of mRNA, tRNA and miRNA.

34. The method of claim 26, wherein the analyte is a chemical compound.

35. The method of claim 26, wherein the analyte is a microorganism or portion thereof.

36. The method of claim 35, wherein the microorganism is a virus, bacteria or fungi.

37. The method of claim 26, further comprising analyzing the analyte.

38. The method of claim 37, wherein analysis comprises detecting a disease or condition.

39. The method of claim 38, wherein the disease or condition is selected from the group consisting of cancer, sexually transmitted infection, pregnancy, fetal defect, status of microbiome and presence of a microorganism.

40. The method of claim 37, wherein analysis comprises detecting a marker for female reproductive management.

41. The method of claim 40, wherein the marker is miscarriage, fetal growth restriction, preterm labor, ruptured membrane, or any combination thereof.

42. The method of claim 37, wherein analysis comprises detecting a marker of female reproductive oncology.

43. The method of claim 42, wherein the marker is of cancer.

44. The method of claim 43, wherein the cancer is ovarian, uterine, or cervical cancer.

45. The method of claim 37, wherein analysis comprises detecting a marker of a female reproductive disorder.

46. The method of claim 45, wherein the disorder is endometriosis or polycystic ovarian syndrome.

47. The method of claim 37, wherein analysis comprises detecting infection.

48. The method of claim 47, wherein the infection is uterine infection.

49. The method of claim 38, wherein analysis comprises determining the presence and strain type of a virus.

50. The method of claim 49, wherein the virus is human papillomavirus.

51. The method of claim 26, wherein the analyte is human chorionic gonadotropin.

52. A device for collecting an endocervical sample comprising:
a) a ring having a central opening; and
b) a membrane coupled to the ring covering the central opening, the membrane having a reagent for performing an assay deposited thereon,
wherein the device is resiliently deformable and flexible.

53. The device of claim 52, wherein the ring is approximately circular or oval in a non-deformed position.

54. The device of claim 52, wherein the ring is sized to be disposed over or proximal to an external os of a uterine cervix or anywhere within the vaginal canal of a human subject.

55. The device of claim 52, wherein the device is composed of a biocompatible polymer.

56. The device of claim 55, wherein the ring is composed of a first biocompatible polymer and the membrane is composed of a second biocompatible polymer.

57. The device of claim 35, wherein the ring and the membrane are composed of the same biocompatible polymer.

58. The device of claim 52, wherein the membrane comprises a plurality of reagents.

59. The device of claim 52, wherein the membrane comprises a functionalized surface.

60. The device of claim 52, wherein the reagent is select from the group consisting of a nucleic acid, amino acid, protein, peptide, hormone, steroid, lipid, antigen, carbohydrate, ion and cofactor.
